(19) United States
(12) Patent Application Publication
(10) Pub. No.: US 2016/0199009 A1
(43) Pub. Date: Jul. 14, 2016

(54) MEDICAL NEEDLE PATH DISPLAY

(71) Applicant: NEEDLEWAYS LTD., Zikhron Yakov (IL)

(72) Inventor: Pinhas GILBOA, Haifa (IL)

(21) Appl. No.: 14/911,107

(22) PCT Filed: Aug. 10, 2014
(86) PCT No.: PCT/IL2014/050719

§ 371 Date: Feb. 9, 2016

Related U.S. Application Data
(60) Provisional application No. 61/864,530, filed on Aug. 10, 2013.
(60) Provisional application No. 61/875,067, filed on Sep. 8, 2013.
(60) Provisional application No. 61/984,898, filed on Apr. 28, 2014.

(51) Int. Cl. A61B 5/00; A61B 6/12; A61B 6/00; A61B 5/06

(52) U.S. Cl. CPC A61B 5/7425; A61B 5/066; A61B 6/12; A61B 6/463; A61B 5/0077

(57) ABSTRACT

A system for facilitating manual alignment of a needle with a planned path of insertion includes first and second cameras supported in fixed spaced relation by a frame such that the optical axes of the cameras form between them an angle of more than 30 degrees, and preferably roughly 90 degrees. A processing system generates video displays for both cameras. A line in each of the video displays corresponding to an input planned path of insertion is determined, and a visual indication of that line is generated on the video displays.

### FIELD AND BACKGROUND OF THE INVENTION

The present invention relates to a system and method for facilitating manual alignment of a needle or the like with a desired path of insertion.

In Interventional Radiology (IR) procedures, needles are inserted percutaneously towards an intrabody target with the aid of medical imaging devices such as Computer Tomography (CT), Magnetic Resonance Imaging (MRI), Fluoroscopes etc. There are devices in the market to assist the physician to perform such procedures. Based on the scanned images of the body, a path from an entry point on the skin to an intrabody target is determined and presenting to the user, allowing him to place a needle and insert it along that path. Some types of known solutions are based on a laser beam projected along that path. Such solutions need to use special needles, having marks embedded at its handle to let the physician place the needle accurately at the beam. Another type of solutions use magnetic tracking sensors embedded at the needle tip, which also need special needles.

There are known attempts to develop medical guiding solutions based on the human stereoscopic perception to guide a medical tool to a target. In those solutions, a virtual target is displayed on two separate displays, one display is projected to the left eye and another projected to the right eye, simulating the parallax needed to introduce depth to a virtual target displayed to the physician. The physician has to bring the tool to coincide with that virtual target. Such solution might work well for a target of a definite point of. Because of the relatively small distance between the eyes, in the magnitude of 65-70 mm, and a minimum convenient accommodation distance of 200 mm, the stereoscopic perception limit the maximum angle between the left and the right eyes to 20 degrees, unless accommodation is difficult to be achieved. At such a small angle, in purpose for the depth perception to work, the vision of each eye need to identify small details and compare them point by point in the both views. The path and the needle, which are both continues lines, lack such details. Each point along a line is identical to another. That might bring ambiguity and inaccuracies, especially at the depth direction. At larger angles required for accurate placement of the needle, the stereoscopic phenomenon cannot be used, and another kind of solution need to be developed.

### SUMMARY OF THE INVENTION

The present invention is a system and method for facilitating manual alignment of a needle or the like with a desired path of insertion.

According to the teachings of an embodiment of the present invention there is provided, a system for facilitating manual alignment of a needle with a planned path of insertion, the system comprising: (a) a first camera having a first field of view and a first optical axis; (b) a second camera having a second field of view and a second optical axis; (c) a frame supporting the first and second cameras in fixed spaced relation such that the first and second optical axes form between them an angle of more than 30 degrees and such that the first and second fields of view overlap; (d) a display screen arrangement comprising at least one screen; and (e) a processing system comprising at least one processor, the processing system being in communication with the first and second cameras to receive video data and in communication with the display screen arrangement to generate a first display displaying video from the first camera and a second display displaying video from the second camera, wherein the processing system is configured to: (i) input data defining a planned path of insertion; (ii) determine a line in each of the first and second fields of view corresponding to the planned path of insertion; and (iii) generate a visual indication of the line in both the first and the second displays.

According to a further feature of an embodiment of the present invention, the planned path and the lines are straight lines.

According to a further feature of an embodiment of the present invention, the frame supports the first and second cameras with the first and second optical axes substantially perpendicular.

According to a further feature of an embodiment of the present invention, there is also provided a registration fixture for attachment to the body of a subject, the registration fixture having a plurality of optical markers, and wherein the processing system is further configured to process the video data from at least one of the first and second cameras to derive a position of the registration fixture relative to the frame.

According to a further feature of an embodiment of the present invention, the processing system is configured to continuously track the registration fixture and to continuously update the visual indication of the line in both the first and second displays according to a current position of the registration fixture.

According to a further feature of an embodiment of the present invention, the registration fixture further comprises at least one contrast marker configured to be visible under at least one volume-imaging modality.

According to a further feature of an embodiment of the present invention, the processing system is further configured to modify the video data by applying local linear magnification to a region of the video adjacent to the planned path, the linear magnification being applied in a direction perpendicular to the line indicating the planned path.

There is also provided according to the teachings of an embodiment of the present invention, a method for facilitating manual alignment of a needle with a planned path of insertion, the method comprising the steps of: (a) providing first and second cameras deployed in fixed spaced-apart relation such that optical axes of the cameras form between them an angle of more than 30 degrees and such that fields of the cameras overlap; (b) inputting data defining a planned path of insertion; (c) determining a line in the field of view of each of the cameras corresponding to the planned path of insertion; and (d) generating a visual indication of the line in a visual display of video from both the first and the cameras.

According to a further feature of an embodiment of the present invention, the first and second cameras are deployed with their optical axes substantially mutually perpendicular.

According to a further feature of an embodiment of the present invention, movement of a registration fixture attached to the body of a subject is tracked, and a position of the visual indication is continuously updated according to the position of the body of the subject.

According to a further feature of an embodiment of the present invention, the registration fixture has a plurality of optical markers, and wherein the tracking is performed by processing video data from at least one of the first and second cameras to derive a position of the registration fixture.

According to a further feature of an embodiment of the present invention, the registration fixture further comprises at least one contrast marker configured to be visible under at least one volume-imaging modality.

According to a further feature of an embodiment of the present invention, video data from the first and second cameras is modified by applying local linear magnification to a region of the video adjacent to the planned path, the linear magnification being applied in a direction perpendicular to the line indicating the planned path.

### BRIEF DESCRIPTION OF THE DRAWINGS

The invention is herein described, by way of example only, with reference to the accompanying drawings, wherein:

FIG. 1 is a general description of the invention;

FIG. 2 is a block diagram of system components;

FIGS. 3a and 3b is drawing of registration fixture used with CT imaging;

FIG. 4 is a description of the planning program;

FIG. 5 is description of the method used to search for the location of the registration fixture on the body of the patient in the planning program;

FIG. 6 is a description of the method used to search for the end of a metal wire in the planning program;

FIG. 7 is path defining part of the planning program;

FIG. 8 is an example for using the system to place the needle along the re-planned path; and

FIG. 9 is a description of the zooming zones using in the invention.

### DESCRIPTION OF THE PREFERRED EMBODIMENTS

The present invention is a system and method for facilitating manual alignment of a needle or the like with a planned path of insertion.

The principles and operation of systems and methods according to the present invention may be better understood with reference to the drawings and the accompanying description.

The present invention facilitates a physician placing a needle on the pre-planned path that leads from an entry-point to an intrabody target. In general, the path is simulated as a thin line superimposed on top of two video images, displaying the volume above the entry-point. The video sources are taken from two different directions. The physician places the needle so that the image of the needle in both videos coincides with the simulated path.

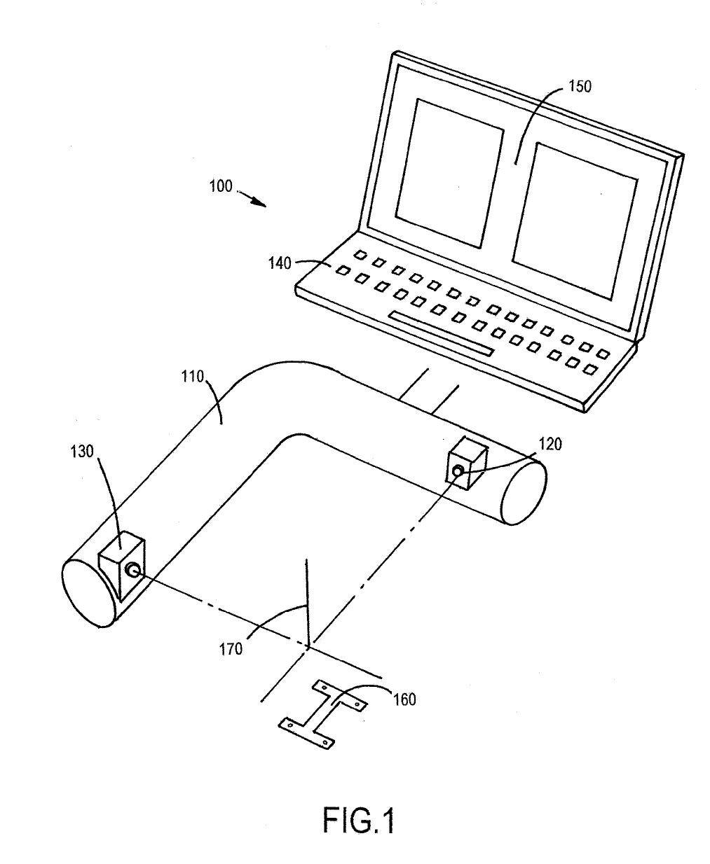

FIG. 1 describes the main components of system 100 used to place needle 170 on a required path. An arm 110 holds two video cameras 120 and 130. A computer 140 is used to receive the output video of the cameras, run software for implanting the simulated path into the video and to display it on the computer screen 150. A Registration Fixture 160 is attached to the patient skin so it can be seen by at least by one of the cameras 120 or 130. The Registration Fixture 160 is used to track the patient position in relation to system coordinates defined by the said two cameras. The technology for tracking the position of an object can be selected from a wide group of well-known solutions such as optical tracking solutions using optical reference markers tracked by one or more camera, magnetic tracking solutions in which a fixture is implemented with one or more flux sensors and electro-magnetic tracking solutions in which a fixture is one or more coils. One U.S. patent example, among many others, for optical tracking technology is U.S. Pat. No. 7,876,942 to Gilboa. U.S. patent examples for electromagnetic tracking technology are U.S. Pat. No. 8,391,952 to Anderson and U.S. Pat. No. 6,833,814 to Gilboa et al. Examples of Magnetic tracking are U.S. Pat. No. 5,744,953 to Hansen, U.S. Pat. No. 8,358,128 to Jensen et al. and U.S. Pat. No. 7,561,051 to Kynor et al.

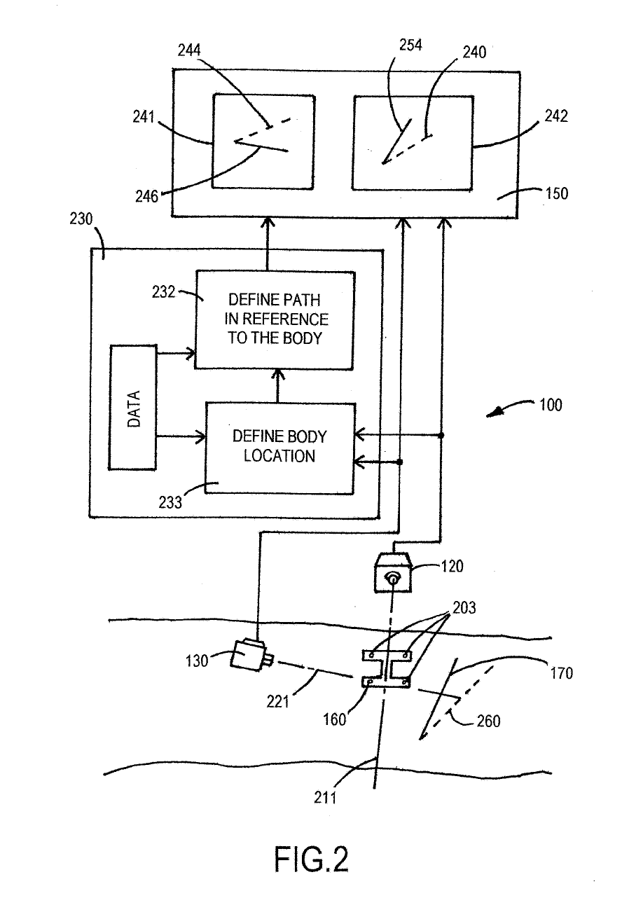

FIG. 2 shows more detailed block diagram of system 100. According to one non-limiting preferred embodiment of the invention, the tracking of Registration Fixture 160 is performed using one or both cameras 120 and 130. For that, the Registration Fixture has identifiable marks such as three or more color dots 203. Other identifiable marks which can also be used are intersecting lines or other shapes that define definite points on top the Registration Fixture and are seen by the at least one of cameras 120 or 130. The video cameras are preferably miniature USB cameras of a type readily commercially available. These types of cameras convert the video image internally to digital form and send it to the computer 140 via a standard USB line.

The pre-planned path data (shown by a dash line 260 in the drawing) is fed to computer 140. Such data includes the location of the identifiable marks 203 in 3D space, the location of the entry-point 205, the location of the target 270 (or the direction towards the target from the entry-point), and optionally the length of the needle shaft or other information describing the geometric shape of the needle.

A software package 230 running on the computer identifies the color dots 130 in the image. From the location of these points in the image, together with their location in the 3D space, the orientation of the camera is calculated using the following:

For v a vector of 4 terms, the location of a point defined in the preplanned space,

R a matrix of 3 by 4 terms defines the translation and rotation of the camera with respect the pre-planned space,

t the transformed of point v into the camera space determined by:

$$[ \begin{matrix}{t}_{x} \\ {t}_{y} \\ {t}_{z}\end{matrix} ] = [ \begin{matrix}{R}_{1 , 1} & {R}_{1 , 2} & {R}_{1 , 3} & {R}_{1 , 4} \\ {R}_{2 , 1} & {R}_{2 , 2} & {R}_{2 , 3} & {R}_{2 , 4} \\ {R}_{3 , 1} & {R}_{3 , 2} & {R}_{3 , 3} & {R}_{3 , 4}\end{matrix} ] \times [ \begin{matrix}{v}_{x} \\ {v}_{y} \\ {v}_{z} \\ 1\end{matrix} ] \qquad ( 1 )$$

The projection p of that point on the focal plan of the camera, where F is the lens' focal length, is

$$[ \begin{matrix}{p}_{x} \\ {p}_{y}\end{matrix} ] = \frac{1}{F \cdot {t}_{z}} [ \begin{matrix}{t}_{x} \\ {t}_{y}\end{matrix} ] \qquad ( 2 )$$

By equations (1) and (2), matrix R can be determine based on known coordinates vᵢ of the identifiable marks and their image coordinates pᵢ, i=1:n. If only one camera is used, n should be at least 4. If two cameras are used, n should be at least 3.

Once matrix R is determined, the projection of path 260, entry-point 205 or any other point defined in the real 3D space can be projected to the computer screen 150 on top of the video images. In FIG. 2, the video image of camera 130 is displayed in video frame 241 on the left side of screen 150. The image of needle 170 is drawn by a solid line 246. The projection of path 260 on the video of camera 130 is drawn by a dash line 244. The video image of camera 120 is displayed in video frame 242 on the right side of screen 150. The image of needle 170 is drawn by a solid line 254. The projection of path 260 on the video of camera 120 is drawn by a dash line 243.

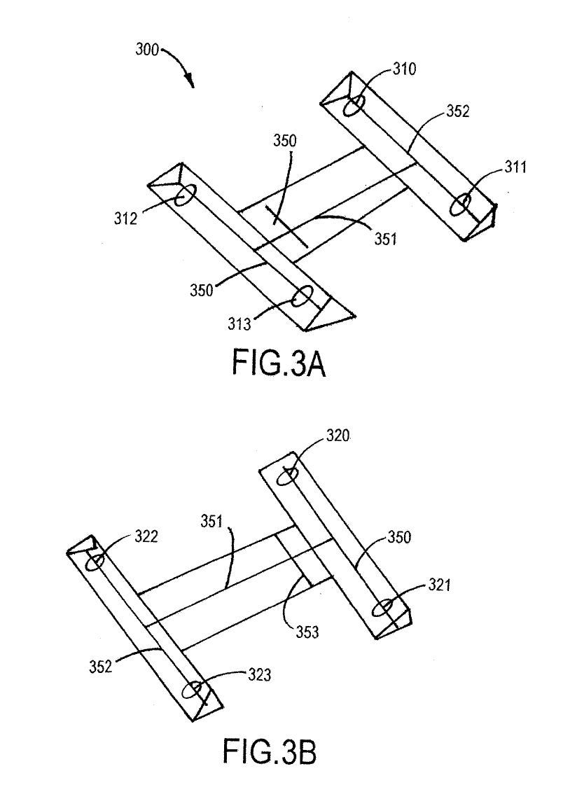

Color dots 203 are embedded on the Registration Fixture 160 at known coordinates, so it is sufficient to determine the location of the fixture in the 3D space to be able to calculate the location of the color dots as well. To enable doing so, fiducial markers, which can be detected by the scanner, are embedded into the Registration Fixture. An example of a Registration Fixture to use with CT imaging modality is shown in FIGS. 3a and FIG. 3b, a solid structure 300 in a shape of the letter H made of bio-compatible plastic material. The arms made of inclined planes, inclined at 45 degrees relative to the H base. Four color dots 310-313 of a one color are embedded on one side of the inclined surfaces and another four dots 320-323 of a different color is embedded on the other side of the inclined surfaces. Four metal wires are embedded into the fixture, wire 350 along a first arm, wire 352 along the opposite arm and wire 351 along the center arm. In addition, a small wire 353 is place asymmetric perpendicularly to wire 352. The metal wires have a high enough contrast when detected in the CT image, allowing easy automatic detection on the surface of the scanned body. Once detected, each of the wires is defined as a vector (origin and direction) in the CT space. Combined together, the position and orientation of the fixture is known, from which the location of the two set of the color dots can also be determined. The structure of the Registration Fixture described in FIGS. 3a and 3b brought herein as an example. Other shapes of contrast objects can be also applicable, such as spheres, disks, rings etc. The material used to form the contrast objects can be made of other materials than metal. Also, for other imaging modalities, the contrast material used to form the said contrast objects need to be one produce high contrast, such as tube filled with oil when using it in MRI. In more generic terms, the reference fixture is configured to have at least one contrast marker configured to be visible under at least one volume-imaging modality, where the phrase “volume imaging modality” is used to refer to any imaging modality allowing imaging of internal structures of the human body.

The technique to determine the required path is closely related to the imaging technology used. In the case of a 3D imaging such as Computer Tomography (CT) or Magnetic Resonance Imaging (MRI), coordinates of the target, coordinates of the entry-point and, if required, the coordinates of fiducial points for registration of the body to the guiding system, are taken directly from the images. It can be simply done because each image point (known as voxel) is directly mapped to a point in space. In the case of 2D imaging such as fluoroscopy, such direct methods are not applicable. Instead, two overlapping images taken at known orientations are used to calculate the 3D coordinates of that object. Each point in the fluoroscopy image represents a vector in space starting at the X-ray source and ending at the image intensifier. For each of the required 3D points of an object in space, its location in both images is marked, defining two vectors intersecting at that object. By calculating the point of intersection, the required point in space is determined.

An implementation of a pre-planning program is brought herein as an example and other implementations are also applicable. Although the following example makes use of the CT imaging device, other scanning modalities can be used as well, with the appropriate needed changes. The program is described herein functionally as a sequence of processes which can readily be implemented by a person having ordinary skill in the art as a software program running on any suitable computer.

The patient is laid on the CT bed. Using the scanned images, the slice coordinate of the target along the bed is identified and the Registration Fixture is attached to the patient skin at or near that coordinate. A volume (spiral) CT scan of the body portion, including the intra-body target and the Registration Fixture, is taken. The scan is sent to a computer running the planning program.

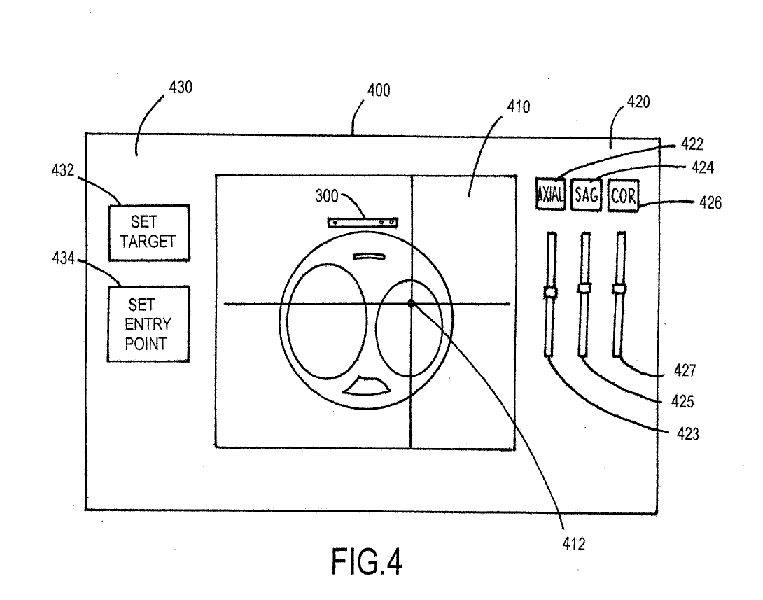

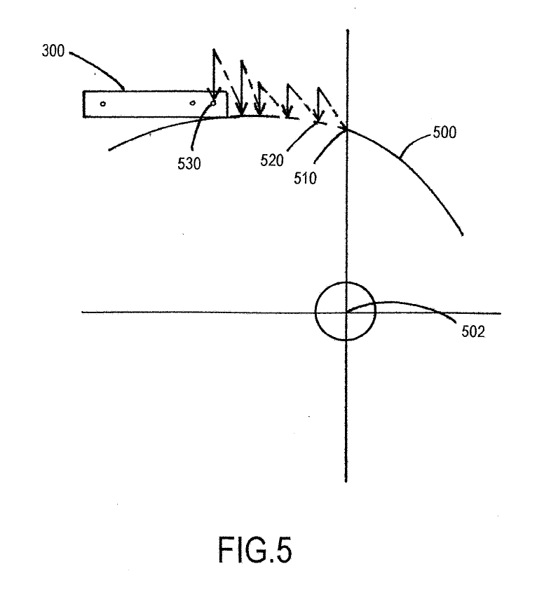

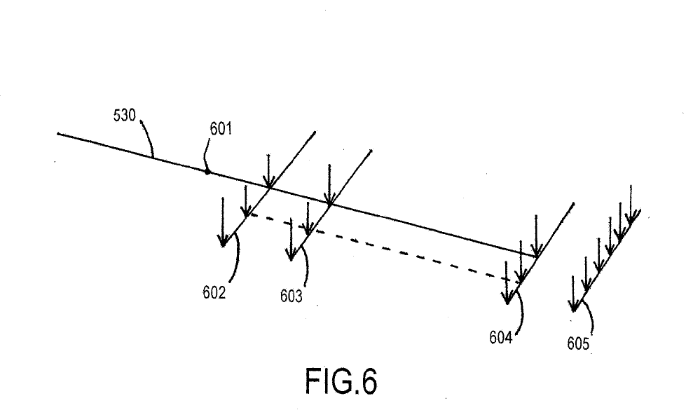

FIG. 4 shows the screen of the planning program. The computer screen 400 is divided into three functional zones, the display zone 410, the display control zone 420 and the program command zone 430. The control zone controls the display. It has three pushbuttons. When the Axial pushbutton 422 is pressed, display 410 shows an axial cross-section of the body, rendered across the center of a 3D cursor location (shown as a cross 412 in the drawing). Similarly, when Sagittal pushbutton 424 or Coronal pushbutton is pressed, sagittal or coronal cross-section is displayed accordingly on display 412. The location of cursor 412 can be changed by pointing to a new location using the computer mouse, or using slider 423 for controlling the axial location or slider 425 for the sagittal location or slider 427 for the coronal location. The operator points at the center of the target and clicks on the ‘Set Target’ command pushbutton. The program stores the coordinates of the cursor as the Target Location coordinates. Next, the program search for the coordinates of the Registration Fixture automatically. FIG. 5 describes how the program searches for the location of Registration fixture 300 on the skin of the patient 500. The program first determines the location of a point on the skin just above target 501, by searching along the path running from the target upwards for the first voxel (CT pixel element) having the density equal to air level. Next, the program searches along the skin for the closest metal wire embedded in the Registration Fixture. This may be done by starting at a certain height above the adjacent voxel 520, and searching downwards for a voxel that has a density value higher than air to indicate where the skin is. During that search, the program also searches for density higher than a certain threshold, indicating metal substance. When a part of the wire is discovered, such as at point 530 in the drawing, that completes this part of the program. Next, the direction of the wire is determined. FIG. 6 describes a general method for searching one of the ends of the wire starting with adjacent point to the first already found point 530 on the wire. Similarly to the previous, the program search for the metal along line 602, which is directing perpendicularly to the wire. Moving to the next adjacent voxel coordinate, search for a metal along line 603, so on until line 605 where metal is not found any metal at all, or it reach the boundary of the scan. The end of the metal wire located at the last found metal coordinates along line 604. Using that method, the program searches for both ends of the first wire and calculates its direction. Metal wire 351 placed perpendicular to the first wire along the skin. Using the methods described, the program looks for its direction and the ends of the other metal wire. The location of the small metal wire 353 determines if the first wire is 350 or 352. The program searches for its location to fully determine the coordinates of Registration Fixture 300 in CT system of coordinates and to determine the color of the dots on each side of the fixture.

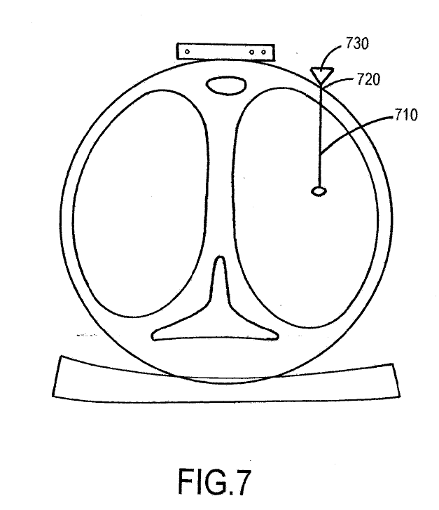

Reference is made now to FIG. 7. The program let the operator to determine the coordinates of the entry-point. The program determines point 720 on the skin, again, as the border between air and higher density along a vector starting from the target. The program draws line 710 connecting the target and that point 720 and an entry-point mark 730 over point 720. The operator drags the arrow mark by the mouse to select the desire path. Once ‘Set Entry Point’ pushbutton 434 is clicked, the program is stored the coordinates of the selected entry-point and the planning phase is ended.

It is essential to this invention, that the cameras would be mutually placed so the line-of-sight 211 of camera 120 and the line-of-sight 221 of camera 130 will have an angle greater than 30 degrees. It more preferably they would place perpendicularly, at 90 degrees, to each other. It is also preferable to be placed so path 260 is about perpendicular to the both line-of-sights. Such an arrangement has the advantages of the highest sensitivity, and allowing convergence and intuitive use of the system.

For bringing the needle onto the path, the practitioner needs to use both video images alternately. It found that when using one of the cameras to move the needle into the path, the user tend to move the needle intuitively perpendicular to the line of sight of that camera. If the line of sights of the cameras are not perpendicular, correcting an error in one of the video images usually produce an error in the other image and vice versa, cause the entire process hardly to converge. Orienting the line-of-sight of the cameras to be substantially perpendicular to each other (90°+/−15°, and more preferably 90°+/−10° solves the problem. Each of the pixels in the video image represents a vector in space, emerging from the pixel through the focal point of the lens and out. Similarly, continues line of pixels at the image represents a plan in space. The path displayed on the first image determines a first plane in space, and the path displayed on the other display determines a second plane. The intersection of the two plans is coinciding with the pre-planned path. Using one of the displays, moving the needle in said plan is displayed in the image as non-moving line. However, on the other video it is changed. The most intuitive use of the system is when these plans located so one plan lay at the direction of the line-of-sight of the physician, and the other plan is perpendicular to his line of sight. In FIG. 1, camera 120 located across the body of the patient in front of the physician, and camera 130 on its left side above about the center of the patient, and the registration fixture attached also at the center of the patient. In that arrangement camera 120 is used also to track the location of the registration fixture, hence the movements of the body of the patient.

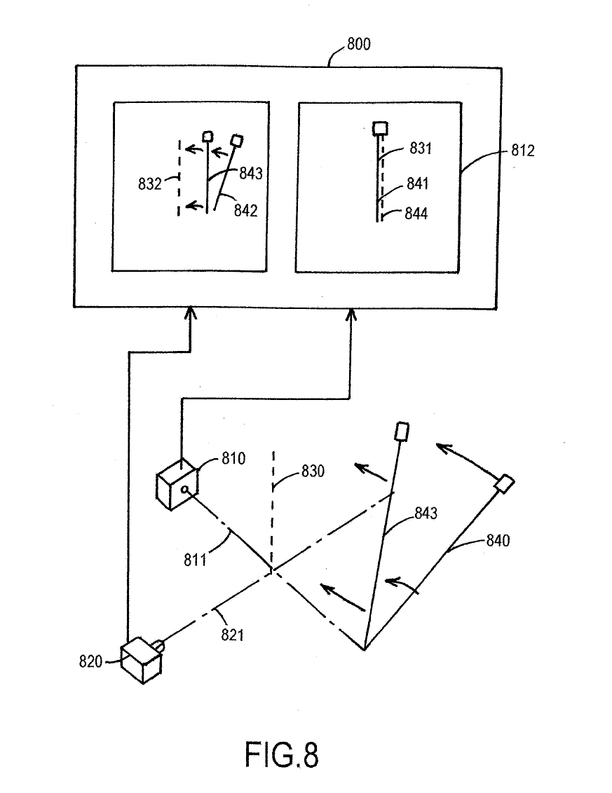

The use of the system is illustrated in FIG. 8. In order to align a needle along a pre-planned path, the needle can conveniently be placed so it appears on the screen parallel to the desired path, and then be moved perpendicularly until it coincides with the path. FIG. 8 demonstrates this procedure. Camera 810 and camera 820 are placed so their lines-of-sight (or “optical axes”) 811 and 821 are mutually perpendicular and also roughly orthogonal to the pre-planned path 830. The preferred orientation of the path of insertion is typically close to vertical, such that a horizontal or near-horizontal layout of the camera support frame typically results in a good approximation to the aforementioned orthogonality. The path is presented as dashed line 831 on the display 812 of the video output of camera 810. The path is presented also as dash line 832 on the display 822 of the video output of camera 820. Suppose that the needle is placed at a first position 840 with respect to the set of said cameras, so it angled to path 830, lies along the line-of-sight 811 of camera 810, and is offset from line-of-sight 821 of camera 820. The image of the needle at this first position as viewed by camera 820 is the tilted line 842 on display 822. However, because both, the needle and the path, are placed along the line-of-sight 811 of camera 810, the image of the needle, as viewed by camera 810 is appear coincide with the path on the video image 812. Next, the needle is rotated to be placed at position 843, parallel to the path 830. The video image of the needle appears at location 845 of video image 822 of camera 820, parallel, but still off path 832. The image 844 of the needle at location 843 on video image 812 of camera 810 remain coincide with path 831 without change. Now, if the needle would be move to be located in coincide with path 830, its image on both displays would be also coincide with the respective line representing the path on the displays. Any of the said changing angles and movement of the needle which are indicated on image 822 is not affect image 812, so image 812 is independent of image 822 with respect of movements of the needle. The same procedure for correcting the location and angles of the needle with respect to the pre-planned path is also true for the other direction, where the needle locate along the direction of the line-of-sight 821 of camera 820. It also works well enough even when the pre-planned path is located offset from the lines-of-sight, and therefore on less perfectly perpendicular planes than was assumed above. Hence, by correcting the position of the needle using one display, moving the needle perpendicularly to the direction of its line-of-sight without effecting the other image and then correcting the position of the needle along the orthogonal direction using the other display, moving the needle perpendicularly to the second line-of-sight, the needle can be brought to be located along the path easily, without confusion that otherwise might arose from dependency of one image with the other. It should be emphasize that when the angle between the above two line-of-sight is significantly less (or more) than 90 degrees, there is increased dependency between the images. In this case, moving the needle perpendicularly to a first line-of-sight of a first camera results in movement of the image of the needle in both displays, resulting in a more awkward and confusing guidance procedure.

The spatial deployment of the components of the system is typically as follows. The patient lies on the bed of the CT imaging system. On one side stands the system made up of the screen 150 and the first camera 120, directed towards the bed and with its optical axis perpendicular to the length of the bed. The second camera 130, supported by an arm of support frame 110, is preferably located roughly over the middle of the width of the bed with its optical axis facing along the length of the bed, perpendicular to the first camera. The registration fixture 160 is preferably attached to the body in a region close to the first camera, while the needle insertion point 170 is preferably in a region further from the first camera. The surgeon preferably stands on the opposite side of the bed from the system. Camera 120, which is also used to track registration fixture 160, is placed so it will be roughly perpendicular to the pre-planned path 260, so its line-of-sight is almost parallel to the length of registration fixture 160. As described and shown on FIGS. 3a and 3b, the color dots may advantageously be placed at 45 degrees tilt so be able to be seen by the camera, but at any given time only one set of the two colors. One set of dots 310 to 313 are seen when the fixture is viewed from one side, and the other set 320 to 323 are seen when the fixture is viewed from the opposite side. Each has its own colors. Based on the location of the asymmetric wire 353, the color of those dots directing towards the camera may be determined. The program is operated to expecting that specific colors in identification of the dots in the video images. If the wrong color is directed towards camera 120, the program will not display the path, avoiding the risk of trying to guide the needle in the wrong part of the body, that might otherwise happen if the system is set up on the wrong side of the body.

The path, as projected on the display, is calculated relative to the Reference Frame, as designated by a registration fixture which is attached to the body of the patient. Hence, when the patient is moved, so the frame is moved and the display of the path is moved as well. As a result, the device described herein is immune to body movements.

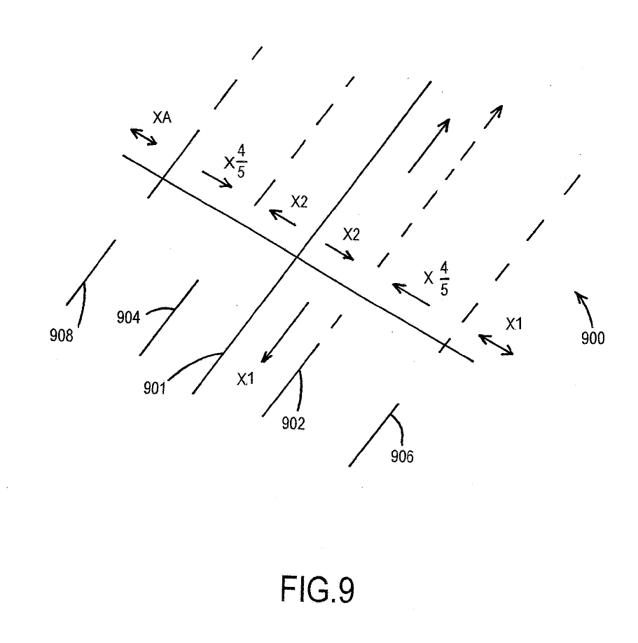

While performing the procedure, the physician typically stands at a distance from the computer screen. Since the needle that in use for most biopsy procedures is thinner than 1.5 mm, it may be difficult to see clearly on the screen. Additionally, in order to avoid masking the image of the needle, the width of the line presenting the planned path is preferably thinner than the appearance of the needle itself, and so is even more difficult to see. Accordingly, according to certain preferred implementations of the invention, zoom is used. However, a simple zoom would cause the loss of valuable information. The active field of view would be narrower and the part of the needle displayed on the screen would be shorter, leading to possible higher angular errors. To overcome these limitations, a non-uniform and directional zoom algorithm is preferably applied. FIG. 9 demonstrates such an algorithm. Line 901 is the indication of the planned direction of insertion displayed on top of video 900. The surrounding pixels on both side of line 901 are zoomed up, but only perpendicular to the line, such that the full portion of the needle originally displayed is still seen on the screen. This type of zoom, which is effectively stretching of the image in one direction, perpendicular to the planned direction of insertion and within defined boundaries, is referred to herein as “local linear magnification”. The zoom of the video image is preferably limited to narrow zone between the boundaries defined in the figure by line 902 and line 904. Inside this zone, every pixel is multiplied (in this example by 2, but other multiplication factors can be applicable too). The multiplication of the pixels necessarily comes at the expense of the surrounding regions, leading to a loss of a portion of the image bordering the magnified strip. To avoid that loss, another two adjacent transition zones are preferably introduced, one shown in the figure between line 902 and line 906, and another from line 904 to line 908. Within this transition zone, the display is preferably contracted perpendicularly to the path in such factor to avoid the loss of the portion of the image. In the example shown in the figure, the width of the contracted transition portion is twice that of the zooming width, so linear magnification (reduction) factor of ⅘ is required. Outside these zones, the image remains the original image.

It might occur during the procedure that the selected entry-point needs to be corrected. A mechanism to change the entry-point, so it still guiding the needle to the selected target, done by the following: getting correction instructions to move the entry-point. According to the new entry-point and the target point recalculate the new path in 3D space. The new path is displayed on the screen. The mechanism for changing the entry-point may include the computer keyboard or the computer mouse. For instance, pushing keys for left, right, forward, backward, up, down, back to original, etc. It also may include doing the same by dragging the image of the entry-point on the screen to the desired new location.

Depending on the kind of procedure and the tooling in use, the path may not necessarily be a straight line. Shaped tools may also be used by presenting the tool shapes (or identifiable parts of the tool) on both screens, so by matching the video image of the tool to the simulated tool on both projected images, the tool is brought to the desire target location also in the angle around its shaft. One example would be an arcuate needle introduced along an arcuate path. In such a case, both the planned path and the displayed lines are generally non-linear.

The optical system could be implemented in reverse, using light projector instead of video camera. In such embodiment, camera 120 and camera 130 are replaced by miniature video projectors. Identical to the former embodiment, a line at the projector focal plan is projected as a plan in space. The intersection of two planes projected by the two projectors determines a line in space. The projected plans are determined by the same mathematics as in the use of camera, determining the location of the pre-planned path on the body of the patient. With respect to the prior art, such projecting system has the advantage of projecting dynamic line in space so, if required, is moves be held at constant position with respect the body of the patient, even when the patient is moved during procedure. In addition, using the ability of video projector to project color images, two different volume of colors can be projected at the two sides of the plan, so let the physician know by the color where to move the needle in purpose to align it with the pre-planned path.

It will be appreciated that the above descriptions are intended only to serve as examples, and that many other embodiments are possible within the scope of the present invention as defined in the appended claims.

What is claimed is:

1. A system for facilitating manual alignment of a needle with a planned path of insertion, the system comprising:
(a) a first camera having a first field of view and a first optical axis;
(b) a second camera having a second field of view and a second optical axis;
(c) a frame supporting said first and second cameras in fixed spaced relation such that said first and second optical axes form between them an angle of more than 30 degrees and such that said first and second fields of view overlap;
(d) a display screen arrangement comprising at least one screen; and
(e) a processing system comprising at least one processor, said processing system being in communication with said first and second cameras to receive video data and in communication with said display screen arrangement to generate a first display displaying video from said first camera and a second display displaying video from said second camera, wherein said processing system is configured to:
(i) input data defining a planned path of insertion;
(ii) determine a line in each of said first and second fields of view corresponding to the planned path of insertion; and
(iii) generate a visual indication of said line in both said first and said second displays.

2. The system of claim 1, wherein said planned path and said lines are straight lines.

3. The system of claim 1, wherein said frame supports said first and second cameras with said first and second optical axes substantially perpendicular.

4. The system of claim 1, further comprising a registration fixture for attachment to the body of a subject, said registration fixture having a plurality of optical markers, and wherein said processing system is further configured to process said video data from at least one of said first and second cameras to derive a position of said registration fixture relative to said frame.

5. The system of claim 4, wherein said processing system is configured to continuously track said registration fixture and to continuously update the visual indication of said line in both said first and second displays according to a current position of said registration fixture.

6. The system of claim 4, wherein said registration fixture further comprises at least one contrast marker configured to be visible under at least one volume-imaging modality.

7. The system of claim 1, wherein said processing system is further configured to modify said video data by applying local linear magnification to a region of said video adjacent to said planned path, said linear magnification being applied in a direction perpendicular to the line indicating the planned path.

8. A method for facilitating manual alignment of a needle with a planned path of insertion, the method comprising the steps of:
(a) providing first and second cameras deployed in fixed spaced-apart relation such that optical axes of said cameras form between them an angle of more than 30 degrees and such that fields of said cameras overlap;
(b) inputting data defining a planned path of insertion;
(c) determining a line in the field of view of each of said cameras corresponding to the planned path of insertion; and
(d) generating a visual indication of said line in a visual display of video from both said first and said cameras.

9. The method of claim 8, wherein said first and second cameras are deployed with their optical axes substantially mutually perpendicular.

10. The method of claim 8, further comprising tracking movement of a registration fixture attached to the body of a subject, and continuously updating a position of said visual indication according to the position of the body of the subject.

11. The method of claim 10, wherein said registration fixture has a plurality of optical markers, and wherein said tracking is performed by processing video data from at least one of said first and second cameras to derive a position of the registration fixture.

12. The method of claim 10, wherein said registration fixture further comprises at least one contrast marker configured to be visible under at least one volume-imaging modality.

13. The method of claim 8, further comprising modifying video data from said first and second cameras by applying local linear magnification to a region of the video adjacent to the planned path, said linear magnification being applied in a direction perpendicular to the line indicating the planned path.
